(19) United States
(12) Patent Application Publication
(10) Pub. No.: US 2026/0033885 A1
(43) Pub. Date: Feb. 5, 2026

(54) HIGH-FREQUENCY APPLICATION METHOD FOR IMPROVING COLLAGEN PRODUCTION AND TISSUE IMPROVEMENT FUNCTION IN SKIN AND SKIN BEAUTY DEVICE USING THE SAME

(71) Applicant: Shenb Co., Ltd., Seoul (KR)

(72) Inventor: Sunyoung KANG, Seoul (KR)

(21) Appl. No.: 19/206,190

(22) Filed: May 13, 2025

(30) Foreign Application Priority Data
Jul. 31, 2024 (KR) 10-2024-0101483

(51) Int. Cl. A61B 18/14; A61B 18/00; A61B 18/12

(52) U.S. Cl. CPC A61B 18/14; A61B 18/1206; A61B 2018/00005; A61B 2018/0047; A61B 2018/00732; A61B 2018/1253; A61B 2018/126

(57) ABSTRACT

Embodiments relate to a high-frequency application method for improving collagen production and tissue improvement function in a skin, and to a skin beauty device using the same, and more specifically, to a high-frequency application method for improving collagen production and tissue improvement function in a skin, and to a skin beauty device using the same, which may induce new collagen production in the skin and improve a basement membrane tissue by cross-and-sequentially applying a monopolar type pulsed high frequency and a bipolar type pulsed high frequency, thereby improving the skin, including the effect of reducing freckles, and increasing skin elasticity and obtaining a lifting effect.

### CROSS-REFERENCE TO RELATED APPLICATIONS

This application claims the priority of Korean Patent Application No. 10-2024-0101483 filed on Jul. 31, 2024, in the Korean Intellectual Property Office, the disclosure of which is incorporated herein by reference.

### BACKGROUND

### Field of the Invention

The present disclosure relates to a high-frequency application method for improving collagen production and tissue improvement function in a skin, and to a skin beauty device using the same, and more specifically, to a high-frequency application method for improving collagen production and tissue improvement function in a skin, and to a skin beauty device using the same, which may induce new collagen production in the skin and improve a basement membrane tissue by cross-and-sequentially applying a monopolar type pulsed high frequency and a bipolar type pulsed high frequency, thereby improving the skin, including the effect of reducing freckles, and increasing skin elasticity and obtaining a lifting effect.

### Description of the Related Art

With the advancement of medicine and medical devices, not only have methods for treating diseases and the body been developed, but interest in skin beauty and regeneration treatments has also been continuously increasing.

Among these, Botox or filler is used as a method to improve skin elasticity and wrinkles, and thread lifting method is used. However, due to side effects such as foreign body sensation in the skin or thread breakage, high-frequency skin beauty treatment that regenerates and regrows collagen in the skin is actively being performed.

Meanwhile, due to the decrease in collagen, skin elasticity rapidly decreases, fine wrinkles appear around the eyes, and a pigmentation phenomenon such as freckles occur, and as the elasticity of blood vessels decreases, redness begins to appear, which occurs due to the influence of the basement membrane among the tissues within the skin.

The basement membrane is a thin, flexible plate-shaped epithelial layer that attaches cells to adjacent connective tissue, prevents malignant cells from invading deeper tissues within the skin, and helps maintain the existing tissue structure during regeneration by moving substances into and out of the connective tissue. Therefore, the main functions of the basement membrane are to support cells and tissues.

As the basement membrane ages, the basement membrane loses the function and becomes weak or destroyed, and thus, freckles occur. In addition, collagen and elastic fibers located beneath the basement membrane decrease, causing the dermis to become thinner, the skin to lose elasticity, and wrinkles to appear.

Meanwhile, skin treatment and improvement methods using high frequency are largely divided into monopolar RF and bipolar RF types depending on the disposition and type of electrodes that generate high frequency.

First, the monopolar type provides high frequency with a single polarity electrode, and has a plate on the opposite side from the treatment area so that the high frequency penetrates the body through the space between the treatment area and the plate to provide energy. Therefore, the monopolar type has the advantage of having a wide and deep area to which high frequency is applied. In particular, through this monopolar, high frequency energy may sufficiently reach the dermis layer to affect and improve skin tissue and internal fat layers. However, when the procedure is not performed properly, there is a risk of burns or sparks, and there is a problem that a test subject feels a lot of pain and experiences increased physical fatigue due to the depth of penetration of the high frequency energy.

Meanwhile, the bipolar type has limited heating and penetration depth compared to the monopolar type, but the bipolar type may apply high-frequency energy intensively to the desired treatment area (mainly the upper part of the dermis), remove skin pores and mild wrinkles, and also achieve a fat burning effect through increased oxygen supply by expanding blood vessels.

However, conventional high-frequency skin medical devices use the monopolar and bipolar types to apply high-frequency waves and perform treatments separately using handpieces and tips, which are cumbersome. Moreover, there is an inconvenience in that the effects of previous treatments become minimal during the process of replacing handpieces, making it difficult to obtain quick treatment and results.

More specifically, when irradiating the treatment area with high frequency using the monopolar type, the high frequency may penetrate deep into the skin, but it is difficult to focus treatment. Accordingly, the heated area through the monopolar type-high frequency cools down during the change from the monopolar to the bipolar, making it difficult to perform proper treatment. This is also the case when performing the opposite type of high frequency treatment, so there is concern that treatments using the monopolar type-high frequency and bipolar type-high frequency may not be performed effectively.

### PRIOR ART LITERATURE

### Patent Literature

(Patent Literature 1) Korean Patent No. 10-1227224 entitled “HIGH-FREQUENCY SKIN TREATMENT DEVICE”

### SUMMARY

The present disclosure is intended to solve the above-described problems, and an object of the present disclosure is to provide a high-frequency application method for improving collagen production and tissue improvement function in a skin, and to a skin beauty device using the same, which may induce new collagen production in the skin and improve a basement membrane tissue by sequentially outputting a monopolar type pulsed high frequency and a bipolar type pulsed high frequency, thereby improving the skin, including the effect of reducing freckles, and increasing skin elasticity and obtaining a lifting effect.

In addition, another object of the present disclosure is to provide a high-frequency application method for improving collagen production and tissue improvement function in a skin, and to a skin beauty device using the same, which may evenly provide a high frequency to the skin layer by providing an optimal ratio of the monopolar type-high frequency and bipolar type-high frequency applied to the skin, thereby promoting new collagen production and thickening and strengthening the basement membrane to obtain the effect of improving freckles and preventing their recurrence.

In addition, still another object of the present disclosure is to provide a high-frequency application method for improving collagen production and tissue improvement function in a skin, and to a skin beauty device using the same, which may simplify treatment and minimize skin pain by irradiating a skin with a monopolar type-high frequency and a bipolar type-high frequency at an optimal ratio by a single handpiece without having to separately prepare monopolar and bipolar type handpieces.

Objects of the present disclosure are not limited to the objects mentioned above, and other objects not mentioned will be clearly understood by those skilled in the art from the description below.

In order to achieve the above-described objects, the present disclosure includes: an electrode unit formed on one end surface of a handpiece that comes into contact with a treatment area, the electrode unit including a first electrode and a second electrode; a high frequency generation unit that transmits high frequency generated in the electrode unit; a conversion unit that converts the first electrode and the second electrode so that the electrode unit applies a monopolar type pulsed high frequency or a bipolar type pulsed high frequency; and a control unit that drives and controls the high frequency generation unit and the conversion unit.

In addition, the first electrode may have a different polarity from the second electrode, and the conversion unit may activate the first electrode to apply the monopolar type pulsed high frequency to the skin, and activate the first electrode and the second electrode to apply the bipolar type pulsed high frequency to the skin.

In this case, the control unit may store a high-frequency output pattern in advance, and in the high-frequency output pattern, the monopolar type pulsed high frequency and the bipolar type pulsed high frequency may be sequentially applied to the skin during treatment through the skin beauty device, and the monopolar type pulsed high frequency and the bipolar type pulsed high frequency may be applied to the skin at a ratio of 2:8.

In addition, a skin stimulation unit may be additionally formed on the end surface of the handpiece where the electrode unit is formed, and the skin stimulation unit may be formed along an edge of the handpiece, have a shape in which the electrode unit is provided in a center, and apply electrical muscle stimulation (EMS) to the skin.

In addition, a cooling unit may be additionally formed on the end surface of the handpiece where the electrode unit is formed, and the cooling unit may be formed along the edge of the handpiece, have a shape in which the electrode unit is provided in a center, and cool the contacted skin surface.

In order to achieve the above-described objects, the present disclosure includes: a driving step of driving a handpiece; a first conversion step of converting an electrode unit provided on one end surface of the handpiece by a conversion unit so that the electrode unit outputs a monopolar type pulsed high frequency; a first high frequency application step of applying the monopolar type pulsed high frequency to a skin by the electrode unit; a second conversion step of converting the electrode unit by the conversion unit so that the electrode unit outputs a bipolar type pulsed high frequency; and a second high frequency application step of applying the bipolar type pulsed high frequency to the skin by the electrode unit.

In this case, a ratio of the number of applications between the monopolar type pulsed high frequency applied in the first high frequency application step and the bipolar type pulsed high frequency applied in the second high frequency application step is 2:8.

According to the present disclosure, it is possible to induce new collagen production in the skin and improve a basement membrane tissue by sequentially outputting the monopolar type pulsed high frequency and the bipolar type pulsed high frequency, thereby improving the skin, including the effect of reducing freckles, and increasing skin elasticity and obtaining a lifting effect.

In addition, according to the present disclosure, it is possible to evenly provide a high frequency to the skin layer by providing an optimal ratio of the monopolar type-high frequency and bipolar type-high frequency applied to the skin, thereby promoting new collagen production and thickening and strengthening the basement membrane to obtain the effect of improving freckles and preventing their recurrence.

In addition, according to the present disclosure, it is possible to simplify treatment and minimize skin pain by irradiating a skin with a monopolar type-high frequency and a bipolar type-high frequency at an optimal ratio by a single handpiece without having to separately prepare monopolar and bipolar type handpieces.

The effects of the present disclosure are not limited to the effects mentioned above, and other effects not mentioned will be clearly understood by those skilled in the art from the description below.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 is a diagram illustrating a configuration of a skin beauty device using a high-frequency application method for improving collagen production and tissue improvement function in a skin according to the present disclosure.

FIG. 2 is an electron microscope photograph of samples for Control Group and Examples of Experimental Example 1 according to the present disclosure.

FIG. 3 is a graph illustrating staining intensity of the samples for Control Group and Examples of Experimental Example 1 according to the present disclosure.

FIG. 4 is an electron microscope photograph of samples for Control Group and Examples of Experimental Example 2 according to the present disclosure.

FIG. 5 is a graph illustrating the strength of a new collagen tissue of the samples for Control Group and Examples of Experimental Example 2 according to the present disclosure.

FIG. 6 is a graph illustrating strength of a mature collagen tissue of the samples for Control Group and Examples of Experimental Example 2 according to the present disclosure.

FIG. 7 schematically illustrates a treatment surface of a handpiece according to another embodiment of the present disclosure.

FIG. 8 schematically illustrates a treatment surface of a handpiece according to another embodiment of the present disclosure.

FIG. 9 is a flow chart illustrating a high-frequency application method for improving collagen production and tissue improvement function in a skin according to the present disclosure.

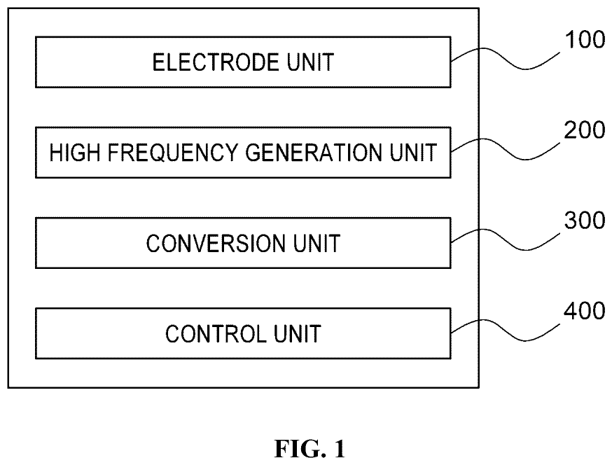

### DETAILED DESCRIPTION OF THE EMBODIMENT

Hereinafter, the exemplary embodiment of the present disclosure will be described with reference to the accompanying drawings and exemplary embodiments as follows. Scales of components illustrated in the accompanying drawings are different from the real scales for the purpose of description, so that the scales are not limited to those illustrated in the drawings.

The present disclosure may have various modifications and embodiments, and specific embodiments are illustrated in the drawings and described in detail.

However, this is not intended to limit the present disclosure to specific embodiments, but should be understood to include all modifications, equivalents, or substitutes included in the spirit and technical scope of the present disclosure. In describing each drawing, similar reference numerals are used for similar components.

When it is said that a component is “coupled” or “connected” to another component, it should be understood that it may be directly connected or connected to that other component, but that there may be other components in between. Meanwhile, when it is said that a component is “directly coupled” or “directly connected” to another component, it should be understood that there are no other components in between.

The terminology used in the present disclosure is only used to describe specific embodiments and is not intended to limit the present disclosure. The singular expression includes the plural expression unless the context clearly indicates otherwise. In the present disclosure, it should be understood that the terms “include” or “have” and the like are intended to specify the presence of a feature, number, step, operation, component, part or combination thereof described in the present disclosure, but do not exclude in advance the possibility of the presence or addition of one or more other features, numbers, steps, operations, components, parts or combinations thereof.

Hereinafter, embodiments of the present disclosure will be described with reference to the attached drawings. The same reference numerals in each drawing represent the same elements. In describing the present disclosure, specific descriptions of related known functions or configurations are omitted in order to avoid obscuring the gist of the present disclosure.

The present disclosure relates to a high-frequency application method for improving collagen production and tissue improvement function in a skin, and to a skin beauty device using the same, and more specifically, to a high-frequency application method for improving collagen production and tissue improvement function in a skin, and to a skin beauty device using the same, which may induce new collagen production in the skin and improve a basement membrane tissue by sequentially applying a monopolar type pulsed high frequency and a bipolar type pulsed high frequency, thereby improving the skin, including the effect of reducing freckles, and increasing skin elasticity and obtaining a lifting effect.

The skin beauty device using the high-frequency application method for improving collagen production and tissue improvement function in a skin according to the present disclosure includes an electrode unit 100, a high frequency generation unit 200, a conversion unit 300, and a control unit 400.

First, the electrode unit 100 includes one or more first electrodes 110 and one or more second electrodes 120. When the first electrode 110 and the second electrode 120 are used as a bipolar type, the first electrode 110 and the second electrode 120 may have different polarities, and when the first electrode 110 and the second electrode output high frequency as a monopolar type, only the first electrode 110 may output a monopolar type-high frequency, or both the first and second electrodes 110 and 120 may output the monopolar type-high frequency.

This electrode unit 100 is formed at the end portion of the handpiece, that is, the end surface that comes into close contact with the skin during skin beauty treatment, and it is preferable that the first and second electrodes 110 and 120 are disposed to cross each other in the up, down, left, and right directions.

Accordingly, the electrode unit 100 performs the role of transmitting the high frequency generated through the high frequency generation unit 200 located inside the handpiece to a treatment area of a contacted test subject, and transmits the high frequency of monopolar or bipolar type to the skin depending on the intention of a practitioner.

Meanwhile, when applying the monopolar type-high frequency to the body of the test subject through the electrode unit 100, a separate electrode plate (not illustrated in the drawings) is provided in addition to the electrode unit 100, so that the electrode unit 100 is in close contact with the treatment area based on the body of the test subject to apply the monopolar type-high frequency, and the electrode plate having a large contact area is positioned on the side opposite to the electrode unit 100. Therefore, the electrode unit 100 and the electrode plate have different polarities so that the monopolar type-high frequency may easily penetrate the body of the test subject.

The high frequency generation unit 200 is a circuit that generates high frequency by receiving a control command from the control unit 400, and the high frequency generated by the high frequency generation unit 200 is transferred to the treatment area through the electrode unit 100. This high frequency generation unit 200 is driven for skin treatment through the skin beauty device, and is driven and controlled together with the control unit 400 through the operation of the practitioner.

The conversion unit 300 selectively converts the high frequency generation unit 200 and the electrode unit 100 to apply the monopolar type-high frequency or the bipolar type-high frequency, and performs the function of converting the electrode unit 100 so that high frequency may be output to the first electrode 110 or the second electrode 120 when the skin beauty device is driven. This conversion unit 300 is provided in the form of a circuit in the handpiece or the main body of the skin beauty device.

The control unit 400 controls the operation of the skin beauty device according to the memory (not illustrated in the drawings) stored in the skin beauty device in advance or the operation of the practitioner (user), and operates the conversion unit 300 so that the electrode unit 100 and the high frequency generation unit 200 output an appropriate type of high frequency and transmit the high frequency to the treatment area. In this case, the operation unit (not illustrated in the drawings) operated by the practitioner operates the type of high frequency, treatment time, or the like to be provided to the practitioner through switches, or the like provided in the beauty device and handpiece that apply high frequency, and the control unit 400 may control the operation of the conversion unit 300 and the electrode unit 100 accordingly.

Meanwhile, the monopolar type-high frequency may be output to the first electrode 110 of the electrode unit 100 through the conversion unit 300 and the control unit 400, or the bipolar type-high frequency may be output through the first electrode 110 and the second electrode 120. In this case, there is a need for the practitioner to obtain the benefits of both the monopolar type-high frequency treatment and the bipolar type-high frequency treatment by using both monopolar type and bipolar type in parallel rather than performing a single type-high frequency treatment as needed.

Meanwhile, when the monopolar type treatment and the bipolar type treatment are simply alternately performed, it is difficult for the practitioner to intentionally obtain the desired skin treatment effect, and when simple cross-treatment is performed, the skin treatment time becomes longer and the pain or swelling of the test subject becomes worse, so effective high-frequency output is required. In this regard, the present applicant has derived the optimal high-frequency application pattern that may efficiently obtain freckle improvement and lifting effects by generating new collagen in the skin and improving tissue (basement membrane) through a number of experiments as follows.

### Experimental Example 1

### Analysis of Changes in the Basement Membrane According to Ratio of Number of High Frequency Applications of Monopolar and Bipolar Types

The test subject was a male Sprague rat (SD Rat) that were 9 weeks old and were subjected to high frequency application. Samples were collected 14 days later (2 weeks) to analyze changes in the basement membrane. The test subject received non-invasive ultrasound treatment, and experiments were conducted by changing the ratio of the number of applications between monopolar type-high frequency and bipolar type-high frequency were applied to the test subject as illustrated in Examples 1 to 11 in Table 1 below. In addition, changes in the basement membrane tissue were confirmed under the same conditions where high frequency was not irradiated as Control Group.

TABLE 1

| Example | 1 | 2 | 3 | 4 | 5 | 6 | 7 | 8 | 9 | 10 | 11 |
|---|---|---|---|---|---|---|---|---|---|---|---|
| Number of applica- | 10 | 9 | 8 | 7 | 6 | 5 | 4 | 3 | 2 | 1 | 0 |
| tions of monopolar | | | | | | | | | | | |
| Number of applica- | 0 | 1 | 2 | 3 | 4 | 5 | 6 | 7 | 8 | 9 | 10 |
| tions of bipolar | | | | | | | | | | | |

In this case, the high-frequency output energy was the same at 2 MHz, 9.6 W (0.96 J), the total high-frequency output time was 100 ms, the high frequency was provided as 10 pulses, and the high frequency off time for each pulse was set to 5 ms. In order to visualize the basement membrane during sample extraction, changes in the basement membrane were confirmed through periodic acid-Schiff stain (PAS stain).

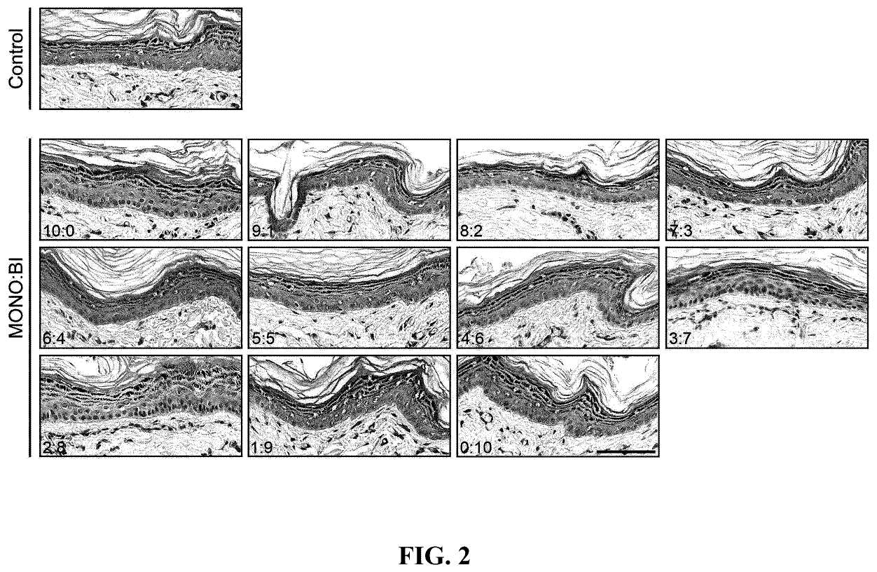

As illustrated in FIG. 2, in Examples 8 and 9 where the ratio between the number of applications of the monopolar and the number of applications of the bipolar was 3:7 and 2:8, respectively, it was confirmed that the basement membrane, which is the skin tissue, was the most improved as the changed basement membrane was more uniform and had increased thickness.

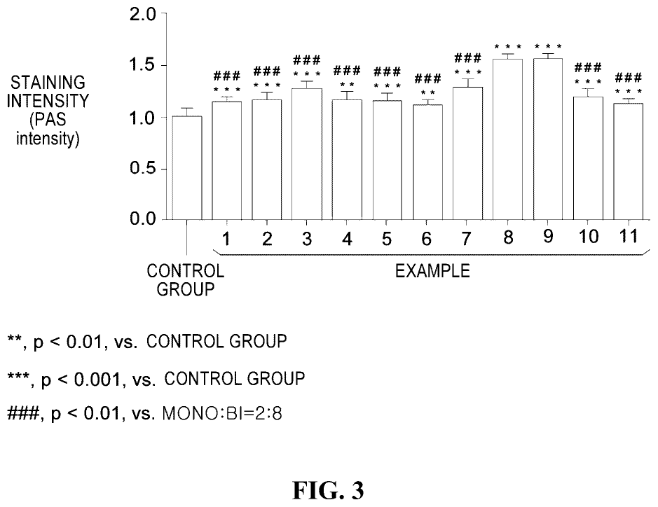

More specifically, as illustrated in FIG. 3, the sample images were analyzed to graph Control Group and Examples by staining intensity, and through this, it was confirmed that Control Group was the lowest, while all Examples showed improved basement membranes compared to Control Group. Among these, it was confirmed that Examples 8 and 9 showed the greatest improvement in basement membranes.

### Experimental Example 2

### Analysis of Changes in New and Mature Collagen Fibers According to Ratio of High Frequency Irradiation of Monopolar Type and Bipolar Type

The experiment was conducted using the same test subject, experimental conditions, Control Group, and Examples as in Experimental Example 1 described above. In this experiment, the changes in collagen fibers were confirmed by staining the sample with Herovici's stain to confirm the fibers of new and mature collagen. Through this staining, new and mature collagen were visualized in different colors, respectively, so that changes in collagen fibers could be confirmed.

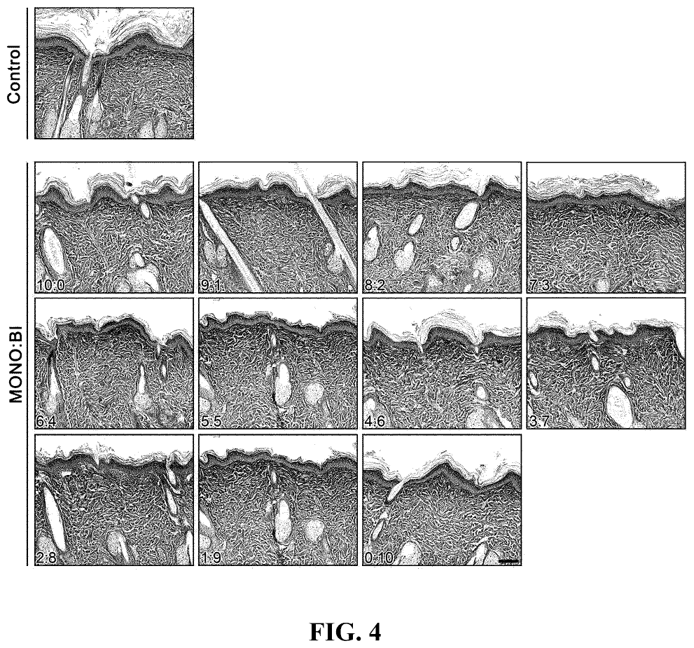

As illustrated in FIG. 4, it was confirmed that the change in collagen fibers significantly increased in Example 9 where the ratio between the number of applications of the monopolar and the number of applications of the bipolar was 2:8.

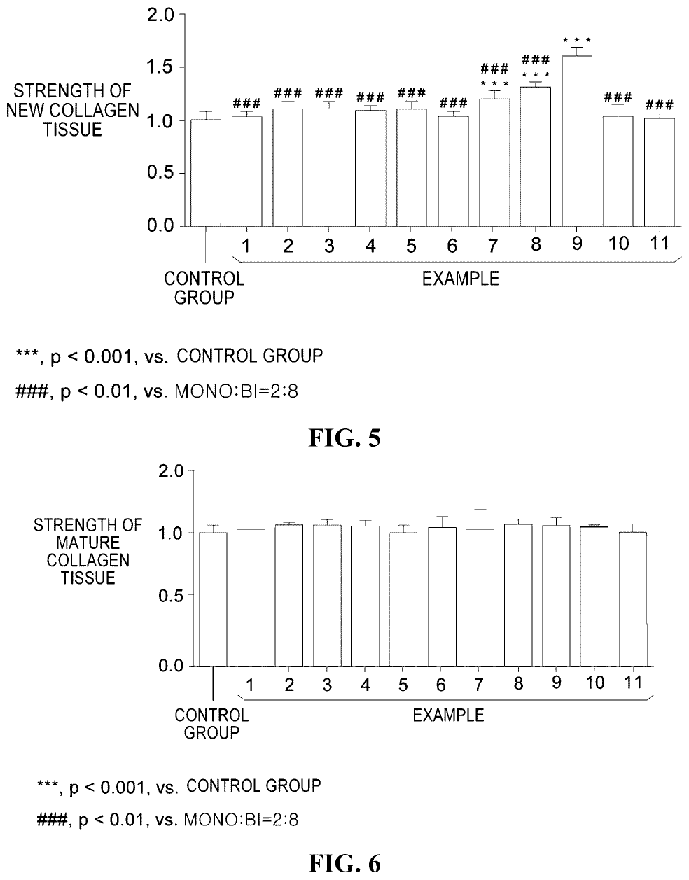

More specifically, as illustrated in FIGS. 5 and 6, the sample images that may confirm changes in new collagen fibers and mature collagen fibers were analyzed and graphed for Control Group and Examples by staining intensity. Through this, it was confirmed that there was no significant difference in mature collagen fibers between Control Group and Examples (see FIG. 6), but that new collagen was significantly increased in Example 9 (see FIG. 5).

As a result of the analysis through these Experimental Examples 1 and 2, it was confirmed that Example 9, in which the monopolar type-high frequency and the bipolar type-high frequency are sequentially mixed and performed on the test subject in a ratio of 2:8, is the most suitable.

That is, through this experiment, it was confirmed that the ratio of 2:8 for the number of high frequency applications of the monopolar type and the bipolar type was the optimal ratio to most efficiently obtain the effects of improving basement membrane tissue and generating new collagen fibers.

Through this ratio of the number of applications, first, the high frequency energy of the monopolar type-high frequency penetrates deep into the skin to improve the fascia layer where the subcutaneous fat layer and basement membrane tissue exist, and then the bipolar type-high frequency induces the production of new collagen fibers located in the epidermal layer and dermal layer, thereby improving the internal skin tissue.

The control unit 400 stores an application pattern for the ratio between the number of applications of the monopolar type-high frequency and the number of applications of the bipolar type-high frequency are applied in accordance with the derived optimal ratio in the memory of the control unit 400 in advance, and may automatically control the monopolar type-high frequency application pattern and the bipolar type-high frequency application pattern to be 2:8 when the practitioner performs the treatment.

In particular, since the high frequency treatment is performed quickly, when the practitioner does not perform a single type of high-frequency treatment by directly operating the device manually through a separate operation unit, the high frequency is automatically output in the pattern of 2:8 in the ratio between the number of applications of the monopolar type-high frequency and the number of the applications of the bipolar type-high frequency to perform the treatment. Therefore, the practitioner may perform the optimal high frequency treatment on the test subject without a separate operation.

In addition, when the practitioner wishes to perform a single type of high frequency treatment, such as the monopolar type-high frequency treatment or the bipolar type-high frequency treatment, the practitioner may perform the desired high frequency treatment by outputting a single type of high frequency through a separate operation.

In addition, as another embodiment of the present disclosure, a skin stimulation unit 500 may be additionally included at one end of the handpiece where the electrode unit 100 is positioned, that is, at the surface that comes into contact with the treatment area during treatment.

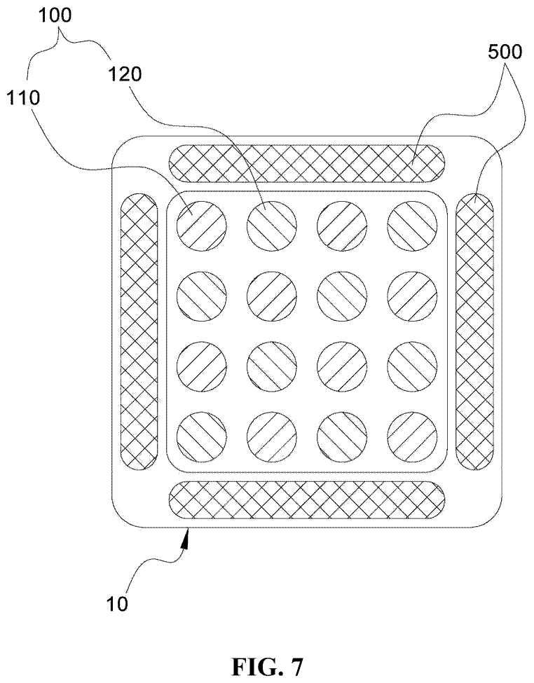

This skin stimulation unit 500 is constituted by an electric muscle stimulation (EMS) pad and is formed along one edge of a handpiece 10 where the plurality of electrode units 100 are positioned as illustrated in FIG. 7. Moreover, the electrode unit 100 is positioned in the center of the skin stimulation unit 500 so that not only treatment through the pulsed high frequency but also stimulation of skin muscles through electric muscle stimulation (EMS) may be performed to further improve skin elasticity.

In this case, the skin stimulation unit 500 may be driven simultaneously with the electrode unit 100 according to the control unit 400 or driven only when the electrode unit 100 stops driving after the treatment is completed, thereby sequentially providing high-frequency stimulation and electrical stimulation to the treatment area to obtain a skin improvement effect.

In addition, as still another embodiment of the present disclosure, a cooling unit 600 may be additionally included at one end of the handpiece where the electrode unit 100 is positioned, that is, at the surface that comes into contact with the treatment area during treatment.

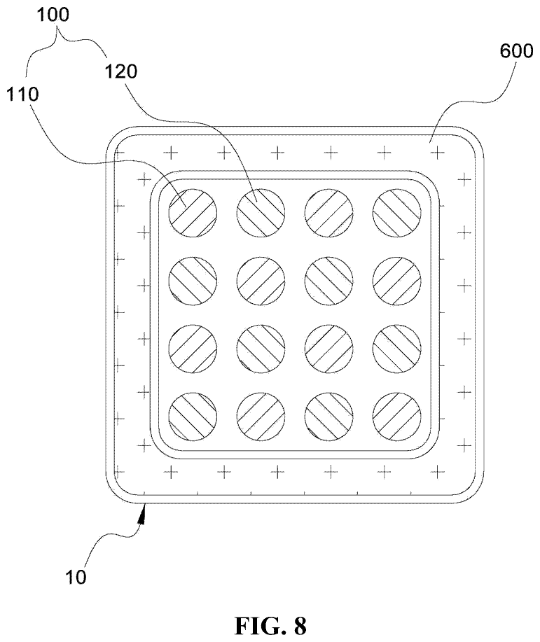

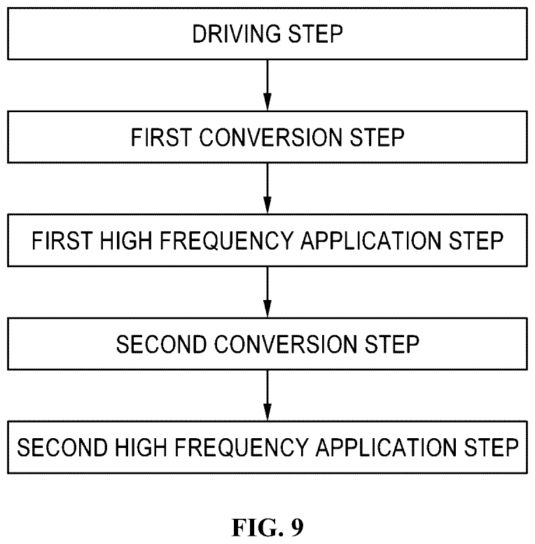

This cooling unit 600 is made in the form of a pad made of a metal material with high thermal conductivity, and is formed along one edge of the handpiece 10 where the plurality of electrode units 100 are positioned as illustrated in FIG. 8 to form a cooling wall on the skin, thereby cooling the skin and absorbing heat generated during the high frequency treatment, as well as having the effect of relieving skin pain and swelling caused by the treatment.

This cooling unit 600 is connected to a cooling block (not illustrated in the drawings) built into the handpiece to quickly absorb heat and cool the treatment area, thereby preventing heat generated in the treatment area from being transferred to other areas.

In this case, it is preferable that the cooling unit 600 is driven simultaneously with the driving of the electrode unit 100 through the control unit 400 to cool the skin surface simultaneously with the treatment through the pulsed high frequency output to relieve swelling and pain, but it is not limited thereto, and the control unit 400 may drive and control the cooling unit 600 according to the operation of the practitioner.

The method of applying high frequency through the skin beauty device described above includes a driving step, a first conversion step, a first high frequency application step, a second conversion step, and a second high frequency application step.

The driving step is a step in which the practitioner operates the skin beauty device according to the intention of the treatment, and the control unit 400 may apply the monopolar type-high frequency and bipolar type-high frequency alternately according to the output pattern described above simultaneously with the driving step.

The first conversion step is a step in which the conversion unit 300 converts the electrode unit 100 so that the electrode unit 100 may output the monopolar type-high frequency.

The first high frequency application step is a step in which the first electrode 110 outputs and applies the monopolar type pulsed high frequency to the treatment area according to the electrode converted through the first conversion step. In this case, in the first high frequency application step, according to the output pattern, 20% of the treatment time to be applied to the practitioner is output and applied as the monopolar type pulsed high frequency.

For example, when the output pattern has the ratio of the monopolar type and bipolar type of 2:8 and the total treatment time is 500 ms, 100 ms including the high-frequency off-time is used in the first high-frequency application step.

The second conversion step is a step in which the conversion unit 300 converts the electrode unit 100 so that the electrode unit 100 may output the bipolar type-high frequency. The conversion unit 300 activates the second electrode 120 so that the electrode unit 100 applies the bipolar type pulsed high frequency to the skin through the first and second electrodes 110 and 120.

After the second conversion step, the second high frequency application step is performed in which the bipolar type pulsed high frequency is applied to the treatment area through the electrode unit 100.

Through the steps described above, the monopolar type pulsed high frequency may be first applied to the skin to penetrate the high frequency energy deep into the skin, and then the bipolar type pulsed high frequency may be applied to induce collagen production within the skin.

The best embodiments have been disclosed in the drawings and specifications. Although specific terms have been used herein, the terms are used only for the purpose of describing the present disclosure and are not intended to limit the scope of the present disclosure as defined in the claims. Therefore, those skilled in the art will understand that various modifications and equivalent other embodiments are possible. Accordingly, the true technical protection scope of the present disclosure should be determined by the technical idea of the appended claims.

What is claimed is:

1. A skin beauty device using a high-frequency application method for improving collagen production and tissue improvement function in a skin, the skin beauty device comprising:
an electrode unit formed on one end surface of a handpiece that comes into contact with a treatment area, the electrode unit including a first electrode and a second electrode;
a high frequency generation unit that transmits high frequency generated in the electrode unit;
a conversion unit that converts the first electrode and the second electrode so that the electrode unit applies a monopolar type pulsed high frequency or a bipolar type pulsed high frequency; and
a control unit that drives and controls the high frequency generation unit and the conversion unit.

2. The skin beauty device according to claim 1, wherein the first electrode has a different polarity from the second electrode, and
the conversion unit activates the first electrode to apply the monopolar type pulsed high frequency to the skin, and activates the first electrode and the second electrode to apply the bipolar type pulsed high frequency to the skin.

3. The skin beauty device according to claim 2, wherein the control unit stores a high-frequency output pattern in advance, and
in the high-frequency output pattern, the monopolar type pulsed high frequency and the bipolar type pulsed high frequency are sequentially applied to the skin during treatment through the skin beauty device, and the monopolar type pulsed high frequency and the bipolar type pulsed high frequency are applied to the skin at a ratio of 2:8.

4. The skin beauty device according to claim 1, wherein a skin stimulation unit is additionally formed on the end surface of the handpiece where the electrode unit is formed, and
the skin stimulation unit is formed along an edge of the handpiece, has a shape in which the electrode unit is provided in a center, and applies electrical muscle stimulation (EMS) to the skin.

5. The skin beauty device according to claim 1, wherein a cooling unit is additionally formed on the end surface of the handpiece where the electrode unit is formed, and
the cooling unit is formed along the edge of the handpiece, has a shape in which the electrode unit is provided in a center, and cools the contacted skin surface.

6. A high-frequency application method for improving collagen production and tissue improvement function in a skin, the high-frequency application method comprising:
a driving step of driving a handpiece;
a first conversion step of converting an electrode unit provided on one end surface of the handpiece by a conversion unit so that the electrode unit outputs a monopolar type pulsed high frequency;
a first high frequency application step of applying the monopolar type pulsed high frequency to a skin by the electrode unit;
a second conversion step of converting the electrode unit by the conversion unit so that the electrode unit outputs a bipolar type pulsed high frequency; and
a second high frequency application step of applying the bipolar type pulsed high frequency to the skin by the electrode unit.

7. The high-frequency application method according to claim 6, wherein a ratio of the number of applications between the monopolar type pulsed high frequency applied in the first high frequency application step and the bipolar type pulsed high frequency applied in the second high frequency application step is 2:8.
